(19) United States
(12) Patent Application Publication
(10) Pub. No.: US 2024/0306942 A1
(43) Pub. Date: Sep. 19, 2024

(54) A SYSTEM, APPARATUS AND METHOD FOR ENHANCING HUMAN SENSORIMOTOR ABILITY

(71) Applicant: PRISM NEURO PTY LTD, Braddon (AU)

(72) Inventor: Elizabeth McGrath, Griffith (AU)

(73) Assignee: PRISM NEURO PTY LTD, Braddon (AU)

(21) Appl. No.: 18/574,138

(22) PCT Filed: Jun. 30, 2022
(86) PCT No.: PCT/AU2022/050683

§ 371 Date: Dec. 26, 2023

(30) Foreign Application Priority Data
Jul. 1, 2021 (AU) 2021902011

(51) Int. Cl. A61B 5/11; A61B 5/00

(52) U.S. Cl. CPC A61B 5/1124; A61B 5/1128; A61B 5/4023; A61B 5/6803; A61B 5/742; A61B 2505/09

(57) ABSTRACT

An apparatus and method for enhancing motor skill ability of a user comprising: a display unit to provide the user with visual images; any, or any combination of a plurality of movement sensing devices for sensing a user's movement response; and a control unit that provides the images to the display unit, records the user's movement responses, and analyses the responses. In use, the visual display unit presents an image of the human body or part thereof to the user and the user is instructed to mentally determine a particular feature of the image and make a corresponding movement in accordance with the feature identified. A sensing device senses the user's movement response. A control unit assesses the response and provides feedback to the user, or alternatively calculates a score for comparison with normative data or to track an individual's progress.

### FIELD OF THE INVENTION

This present invention relates generally to a system, an apparatus, and a method for enhancing human sensorimotor ability. In particular, but not exclusively, training undertaken on the apparatus can be used to improve or rehabilitate a user's motor skill ability. Embodiments of the present invention can also be used as a tool for monitoring the improvements in sensorimotor ability.

### BACKGROUND OF THE INVENTION

The sensorimotor system is a part of the nervous system, concerned with the conscious and unconscious control of motor skill ability. The sensorimotor system comprises sensing mechanisms, sensory neurons carrying sensory information to the brain, neural processing, and motor neurons that carry movement signals to muscles. The somatosensory system is a critical component of the sensorimotor system.

The brain combines sensory information with retained knowledge about the body to understand the body's position in space, which is the foundation of sensorimotor ability. The somatosensory component of the sensorimotor system uses mechanoreceptors in the body to provide information about joint position to the brain. The brain's retained intrinsic and extrinsic model of the body allows it to understand the size and shape of the body. The integration of the joint position information, with body size and shape information allows the brain to understand the position of the body in space. The ability to accurately calculate body position in space is critical for motor skill ability.

Motor imagery, or mental practice of physical tasks through imagined movement, is well established in education, psychology, sport, and medicine. It is an intervention proven to accelerate motor learning and improve motor skill ability. When undertaking a motor imagery task the user incorporates explicit (conscious) and implicit (unconscious) components of the sensorimotor schema without moving the body segments involved.

Repetitive active movement therapy has been shown to be effective in the rehabilitation of motor control. As an example, wobble board training, training that involves balancing on an unstable platform, has been shown to improve motor skill ability. The small repetitive movement adjustments made by a user as required to maintain their balance on a wobble board repetitively stimulate the mechanoreceptor components, and associated neural pathways, of the somatosensory system.

Current methods for rehabilitating or improving motor skill ability rely on either a mental component, such as motor imagery, or a physical component, such as wobble board training. The present inventors have found that combining a mental imaging task with an active physical task response would greatly improve the efficiency and effectiveness of such rehabilitation or improvement efforts by engaging both implicit and explicit components of the sensorimotor system in parallel with movement therapy rather than in series as is the current approach.

Multisensory stimulation through the addition of sound to somatosensory stimulation can further enhance the motor skill improvement process. The somatosensory cortex of the brain responds to signals from mechanoreceptors in the body and processes this input to contribute to motor signals necessary for skilled movement. It has been shown that sound stimulus can also evoke responses in the somatosensory cortex of the brain and combining these two mechanisms of brain stimulation further enhances the effectiveness of motor skill improvement.

### SUMMARY OF THE INVENTION

Herein is described an apparatus for enhancing motor skill ability of a user by combining a repetitive series of motor imagery tasks involving specific body parts, with the active movement by the user of the corresponding body parts. The invention comprises: a display unit to provide the user with a series of visual images, a sensing device for sensing a user's movement response which is made as a result of their mental analysis of each displayed image (the mental task), and a control unit that provides the images to the display unit, records the user's response, and analyses the response compared with the image. In use, the visual display unit presents an image of a human body component (for example, an image of a human body joint in a random orientation, with a random degree of flexion or extension or rotation) to the user and the user is instructed to mentally determine a particular feature of the image (for example, the user may be instructed to identify the human body joint displayed in the image and whether it is right-sided or left-sided) and make a corresponding movement in accordance with the feature identified (for example, the user may be instructed to rotate the right ankle when the user identifies an image containing a right ankle). A sensing device senses the user's movement response. A control unit assesses the response and provides feedback to the user or may store the user response for later analysis. The control unit may also calculate a score that can be stored and used to monitor improvements in motor skill ability

An accessory to the apparatus is a speaker system used to deliver auditory stimulation to the user during a training session to further enhance the motor skill ability of the user. The speaker system is usually but not necessarily in the form of wearable headphones. The control unit, through either a wired or wireless connection to the speaker system, delivers the sound signal to the speaker system.

In an exemplary embodiment the visual display unit comprises a video screen (whether a large stationary screen, a computer screen, a tablet computer screen, or a mobile device screen) for displaying an image to be used in a mental task performed by the user; the sensing device comprises a movable ovoid curved platform being rotatable by the user about an axis of rotation; in use, the user stands astride the axis of rotation of the movable ovoid curved platform in an even weight-bearing stance such that the long axis of each foot is aligned with the axis of rotation of the movable ovoid curved platform, the movable ovoid curved platform incorporates a motion sensor system that indicates movement of the movable ovoid curved platform for sensing a user ankle movement response made as a result of completing the mental task; and a control unit comprising a computing device with a connection, whether wired or wireless, to each of the display unit and the sensing device, for sending images to the display unit and receiving a user response from the sensing device, the user response being utilised to signal the advance of the visual images through a pre-determined or random sequence, with the computing device recording and assessing the user responses and generating feedback on the basis of the user responses.

In an embodiment the sensing device comprises joysticks. Joysticks would typically but not necessarily be used if the user was required to respond to the mental task by moving their wrist.

In an embodiment the sensing device comprises handheld motion sensors. Handheld motion sensors would typically but not necessarily be used if the user was required to respond to the mental task by moving upper body joints such as the elbow or the shoulder.

In an embodiment the sensing device comprises a wearable array of motion sensors. A wearable motion sensor array could allow positioning of the sensors to capture movement responses of the user for any body part.

In an embodiment the sensing device comprises optical motion sensing, which could be achieved through an array of optical sensors or through video motion capture. Optical movement sensors could allow capture of movement responses by the user for any body part.

In an embodiment the sensing device comprises a movable ovoid curved platform being rotatable about an axis of rotation and with the movable ovoid curved platform being equipped with sensors to detect a user's ankle motion response, in combination with any or all of joysticks, handheld motion sensors, a wearable array of motion sensors, or optical motion sensors.

In an embodiment the sensing device comprises a portable platform incorporating two plates that are each rotatable about their own axis of rotation with the two axes of rotation usually but not necessarily being parallel to each other and with the base unit being equipped with sensors to detect a user's ankle motion response through movement of the rotatable plates.

In an embodiment the sensing device comprises a portable platform incorporating two plates that are each rotatable about their own axis of rotation with the two axes of rotation usually but not necessarily being parallel to each other and with the base unit being equipped with sensors to detect a user's ankle motion response through movement of the rotatable plates, in combination with any or all of joysticks, handheld motion sensors, a wearable array of motion sensors, or optical motion sensors.

In an embodiment the visual display unit comprises a wearable head mounted display (whether a virtual reality headset, augmented reality glasses, or other wearable display) such that the images displayed to the user by the visual display unit may be displayed to both eyes together or selectively to only one eye at a time.

In an embodiment the visual display unit comprises a video screen (whether a large stationary screen, a computer screen, a tablet computer screen, or a mobile device screen) in combination with an accessory in the form of a wearable head mounted display (whether augmented reality glasses, or other wearable display) such that the wearable display may be selectively configured by the control unit to allow the images displayed to the user by the visual display unit to be displayed to both eyes together or selectively to only one eye at a time.

### BRIEF DESCRIPTION OF THE ACCOMPANYING DRAWINGS

The preferred embodiments of the present invention will now be described, by way of example only, with reference to the accompanying drawings, in which:

FIG. 1 is a schematic of a system comprising a visual display unit, an accessory to the visual display unit in the form of augmented reality glasses, a sensing device comprising a movable ovoid curved platform with motion sensor, and a control unit, for implementing an embodiment of the present invention;

FIG. 2 shows a user operating the apparatus of FIG. 1.

FIG. 3 shows a schematic of a system comprising a visual display unit, a sensing device comprising a portable platform incorporating two plates that are each rotatable about their own axis of rotation each equipped with a motion sensor, an accessory to deliver sound stimulation to the user in the form of headphones, and a control unit, for implementing a further embodiment of the present invention.

### DETAILED DESCRIPTION OF EXEMPLARY EMBODIMENTS OF THE INVENTION

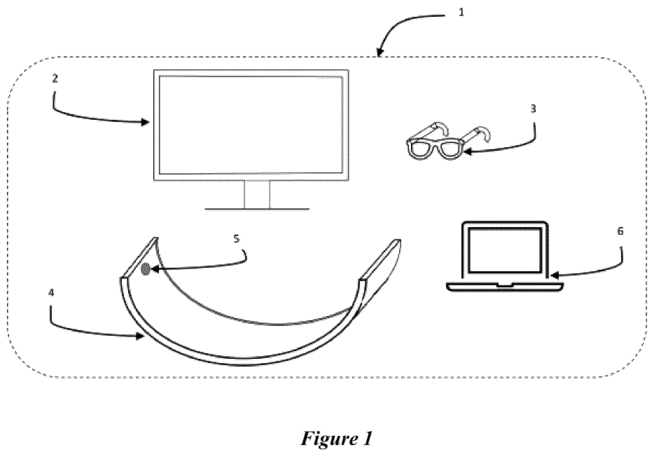

With reference to FIG. 1 there is shown a schematic illustration of a system 1 implementing an apparatus in accordance with an embodiment of the invention. As will be described in detail in subsequent paragraphs, the apparatus enables improvement and rehabilitation of motor skill ability. Furthermore, the apparatus enables monitoring of improvements or decline in these skills.

According to the illustrated embodiment, the apparatus in FIG. 1 comprises a visual display unit 2 for presenting images to a user, and an accessory to the visual display unit being augmented reality glasses 3 worn by the user. A base unit 4 is equipped with a motion sensor 5 for registering user movement responses associated with the mental imaging task (as will be described in more detail in subsequent paragraphs). A control unit 6 is provided for sending visual images to the visual display unit 2, receiving sensor output data from the base unit 4 as well as for generating control signals (e.g. for controlling the display of the augmented reality glasses 3, etc). According to the illustrated embodiment, the visual display data, sensor data, and control signals are wirelessly communicated using Bluetooth connectivity, although these communications can be made using any suitable wired or wireless connection or communication protocol.

The sensing device of FIG. 1 comprises a base unit being a rectangular platform with a symmetrical ovoid curvature of its long axis with respect to a perpendicular axis of rotation that is aligned with the central short axis of the platform such that the resulting curved rectangle contacts the horizontal plane along the axis of rotation and is free to rotate in either of two directions about this axis. The curved platform is sized to allow a range of different sized users to stand in a comfortable stance with feet approximately shoulder width apart and can also accommodate a wider stance for a more challenging experience. The curved platform may be constructed of wood but is not limited to this material. In an embodiment using wood, the wood is heated to a pliable state then held in place over a solid curved form of the desired radius until cooled. The inner, or concave, surface of the curved platform is ideally covered with a textured material such as textured rubber matting to prevent slipping and to stimulate the skin tactile receptors on a user's bare feet. The base unit is equipped with a motion sensor that is able to detect movement of the curved platform.

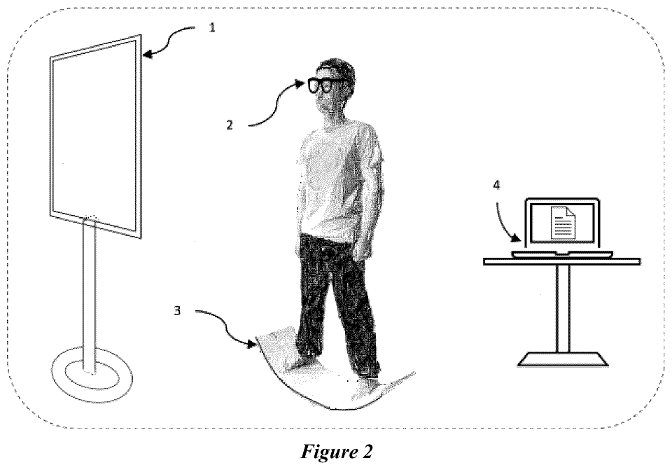

With reference to FIG. 2 a user is shown using the apparatus of FIG. 1. Prior to beginning a session on the apparatus, a user is instructed on the mental task to be accomplished and the resulting response to provide. For this example the user is instructed to identify the body joint shown in a displayed image and whether it is right-sided or left-sided, and when a right-sided ankle is identified the user is instructed to respond by rotating their right ankle outward to initiate a stepping down movement on the right foot, and when a left-sided ankle is identified the user is instructed to respond by rotating their left ankle outward to initiate a stepping down movement on the left foot. The user is further instructed that they should return to a central balanced position following a movement response in order to continue with the session. A session consists of a series of images, with each image requiring a mental task to identify the body joint, a corresponding movement response, and a return to a balanced central position. In addition to the therapeutic benefit gained from linking of the mental task with an active movement, the repetitive balance requirement between responses also provides a therapeutic benefit.

Again, with reference to the exemplary embodiment of the invention in FIG. 2 in use, to begin a session, a pre-loaded therapy paradigm that corresponds to the instructions given to the user is selected on the control unit, the control unit is notified of the start of a new session using the selected therapy paradigm, this notification may be through a button push, a verbal command, a mouse click or other means. Following the notification to the control unit, the user must stand in an even weight bearing stance on the movable platform such that the curved platform is held in a balanced position by the user for a defined period of time. After the balance is held for the defined period of time an image of the human body, or part thereof, is displayed to the user. The user responds to the image according to the instructions provided. The control unit registers the user response and records the time taken to respond, the control unit may also visually or audibly notify the user that their response was recorded, the control unit compares the user response against the displayed image and assesses it as correct or incorrect according to the selected therapy paradigm, the user may be provided with feedback on whether or not their response was correct. When the user has returned the platform to a balanced position the control unit displays the next image in the therapy session. At the end of the session the control unit may store the user results and may further process the results to calculate a score that can be used for comparison with prior or subsequent sessions to monitor a user's improvement.

Again, with reference to the exemplary embodiment of the invention in FIG. 2 in use, a pre-loaded therapy paradigm may include the selected display of images to one eye only or to both eyes. For example, the paradigm may display images of right-sided body joints to the right eye only and images of left-sided body parts to the left eye only, or any combination thereof. This is achieved through the control unit communicating with, and sending control signals to, the wearable accessory display device in the form of augmented reality glasses. In the example the accessory would be configured to block the vision in one eye to allow the other eye to see the visual image on the display unit.

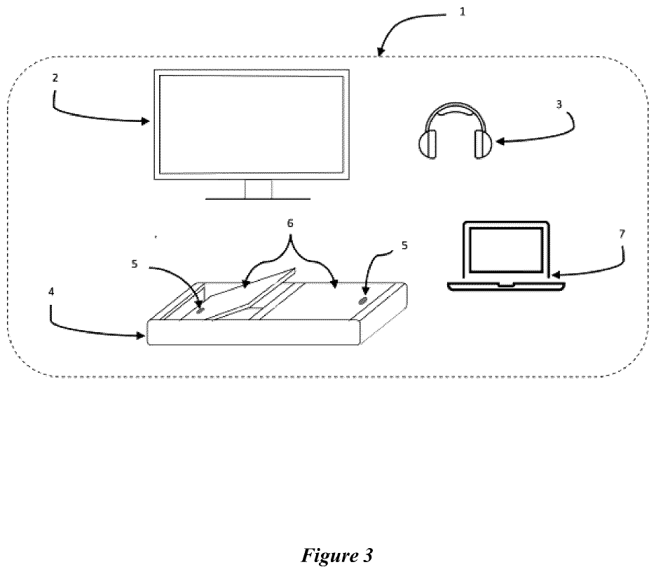

With reference to the exemplary embodiment of the invention in FIG. 3 the control unit 7 communicates wirelessly with the visual display unit 2, and the sensing device 4. The control unit in this embodiment is additionally communicable with a speaker system in the form of headphones 3 worn by the user that deliver sound stimulus to the user during the training session. According to the illustrated embodiment, the sensing device 4 comprises a portable platform incorporating two plates 6 that are each rotatable about their own axis of rotation, and with each being equipped with a motion sensor 5.

There may be other embodiments, in addition to those specifically described here, that would allow for the combined mental imaging task with an active physical response for sensorimotor improvement. The invention is not limited by the specific descriptions included herein.

The control units described here are such that they are able to acquire, store, process, display and send data and information. The data and information are not limited in form and there are many possible mechanisms for the different activities of the control unit that may be applied to the data and information. These control units may be comprised of multiple components and are not limited to a specific configuration. While various embodiments of the present invention have been described above, it should be understood that they have been presented by way of example only, and not by way of limitation. It will be apparent to a person skilled in the relevant art that various changes in form and details can be made therein to suit different situations without departing from the spirit and scope of the present invention. Thus, the present invention should not be limited by any of the above-described exemplary embodiments.

1. An apparatus for enhancing motor skill ability of a user through multimodal sensorimotor stimulation by combining a repetitive series of motor imagery tasks involving mental manipulation by the user that relies on the user's internal body model with the user's active movement of body parts that are linked to the motor imagery task as determined by the user, said apparatus comprising:
an unstable platform that features an element which is moveable by the user, where said element comprises the entire platform or a part thereof, with the user maintaining active standing balance on said unstable platform wherein the user's sensorimotor system is being further stimulated by the act of balancing on said unstable platform while the user undertakes the motor imagery tasks and active movement of body parts, and in between said tasks;
said apparatus further comprising a display unit to provide the user with a visually acquired motor imagery task;
a motion sensor system for sensing the user's movement response which is made as a result of the user's mental performance of each motor imagery task that involves sensing the user's movement of the movable element of the unstable platform and sensing other user movements; and
a control unit that provides the motor imagery tasks to the display unit that receives the motion sensor system output allowing the user's movement responses to be recorded.

2. An apparatus as defined in claim 1, where said apparatus further comprises:
a means for the presentation of a series of random visual images of body parts in random orientations to the user;
a means for instructing the user to identify, through the use of motor imagery relying on the user's internal body model, the body part presented in the image and whether it is from the right or left side of a body;
a means for instructing the user to move the moveable part of the unstable platform on the same side of the user's body that corresponds to the side of the body the user identified in the motor imagery; and
a means for instructing to the user to move the moveable part of the unstable platform on the opposite side of the user's body as compared to the side of the body the user identified in the motor imagery.

3. The apparatus as defined in claim 1, wherein said unstable movable platform is a curved platform.

4. An apparatus as defined in claim 3, wherein a user can stand astride the axis of rotation of the unstable movable curved platform in an even weight-bearing stance such that the long axis of each foot is aligned with the axis of rotation of the unstable movable curved platform.

5. An apparatus as defined in claim 4, wherein the unstable movable curved platform is in the shape of an ovoid.

6. An apparatus of claim 5, wherein the unstable moveable curved platform is made of wood.

7. The apparatus of claim 1, wherein the unstable movable platform comprises a portable platform incorporating two plates that are each rotatable about their own axis of rotation with the two axes of rotation usually but not necessarily being parallel to each other and with the platform being equipped with motion sensors to detect a user's motion response through movement of the rotatable plates.

8. The apparatus of claim 1, wherein the motion sensor system further comprises handheld motion sensors that would typically be used if the user were required to respond to the mental motor imagery task by moving upper body joints such as the elbow or the shoulder.

9. The apparatus of claim 1, wherein the motion sensor system further comprises a wearable array of motion sensors that can allow positioning of the sensors to capture movement responses of the user for any body part.

10. The apparatus of claim 1, wherein the motion sensor system further comprises optical motion sensing, which could be achieved through an array of optical sensors or through video motion capture for allowing capture of movement responses by the user for any body part.

11. An apparatus as defined in claim 1, wherein the control unit is able to register the user response and record the time taken from the presentation of the visual image to the user's movement response, notify the user either visually or audibly that the response was recorded, compare the user response against the displayed visual image and assesses it as correct or incorrect according to the instructions provided to the user and provide feedback to the user whether the response was correct.

12. An apparatus as defined in claim 1, wherein the control unit displays the next random image in the therapy session after the user has returned the motion sensing device to a neutral position.

13. An apparatus as defined in claim 11, wherein the control unit stores the user results at the end of a therapy session and process the results to calculate a score that is based on the time taken for each user movement response, and the assessment by the control unit of whether the responses were correct when compared with the instructions and images provided to the user, and that can be used for comparison with prior or subsequent sessions to monitor a user's improvement.

14. An apparatus as defined in claim 1, wherein the visual display unit comprises a wearable head mounted display such that the random images displayed to the user by the visual display unit may be displayed selectively to only one eye at a time in order to provide a challenge to the user's sensorimotor system by limiting access to visual information.

15. An apparatus as defined in claim 14, wherein the wearable head mounted display is a virtual reality headset, augmented reality glasses or other wearable display.

16. An apparatus as defined in claim 1, further comprising a speaker system used to deliver auditory stimulation from the control unit to the user during a training session to further enhance the motor skill ability and the therapeutic effect via multisensory stimulation by the addition of particular sound to somatosensory stimulation.

17. An apparatus as defined in claim 16, wherein the speaker system is in the form of wearable headphones and the said control unit, through either a wired or wireless connection to the speaker system, delivers the sound signal to the speaker system.

18. A method of enhancing motor skill ability of a user through multimodal sensorimotor stimulation, by combining a repetitive series of motor imagery tasks involving mental manipulation and imagined movement of specific body parts by the user that relies on the user's internal body model, with the active movement of body parts that are linked to the motor imagery task as determined by the user, with the user maintaining active standing balance on an unstable platform wherein the user's sensorimotor system is being further stimulated by the act of balancing on said unstable platform while the user undertakes the motor imagery tasks and active movement of body parts, and in between said tasks, said method comprising the steps of presenting a series of random visual images of random body parts or components thereof of a human body in a random orientation to the user via a visual display unit, providing instructions to the user with the effect of, the user mentally determining a particular feature of the image, the user making a corresponding active body movement in accordance with the feature identified, sensing the user's movement response using a motion sensing device for sensing the user's movement response which is made as a result of the user's mental analysis of each displayed random image, assessing the said response using a control unit that provides the images to the display unit, which records the user's response, analyse the said response compared with the random image and provide instructions and feedback to the user.

19. The method as defined in claim 18, wherein said motion sensing device is mounted on said unstable movable platform or in some other location.

20. A method according to claim 18, further comprising the step of calculating a score that can be stored and used to monitor improvements in motor skill ability.

21. (canceled)
